(19) United States
(12) Patent Application Publication
(10) Pub. No.: US 2007/0003482 A1
(43) Pub. Date: Jan. 4, 2007

(54) Ultrasound contrast agents for molecular imaging

(75) Inventors: Ties Van Bommel, Horst (NL); Nicolaas Petrus Willard, Valkenswaard (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC, N.V., EINDHOVEN (NL)

(21) Appl. No.: 10/596,920

(22) PCT Filed: Jan. 6, 2005
(86) PCT No.: PCT/IB05/50075

§ 371 Date: Jun. 29, 2006

(30) Foreign Application Priority Data
Jan. 15, 2004 (EP) 04100117.3

(51) Int. Cl. A61K 49/00; C07K 16/46

(52) U.S. Cl. 424/9.5; 977/929; 530/391.1

(57) ABSTRACT

The present invention provides a new type of contrast agent which comprises metal nano-particles as well as the method of imaging therewith. The metal nano-particles are stable, biocompatible and can be coupled to bio-target-specific molecules for targeted visualization.

The invention relates to a new type of contrast agent (UCA) for sonography and ultrasound imaging especially Molecular Imaging as well as the method of imaging therewith.

In the last 15 years a number of safe and practical ultrasound contrast agents (UCAs) have been developed. Most of these are based on gas-filled microbubbles, which enhance Doppler signals. The shell is either protein, lipid, surfactant or polymer and they have a particle size of a few microns, allowing them to pass through the lungs. Furthermore, UCAs based on bubbles are compressible, which causes a non-linear behaviour, namely they resonate at specific frequencies of ultrasound typically employed in clinical studies.

Although microbubble agents have been proven useful, they have significant limitations, such as their limited lifetime and the difficulties of targeting them to specific organs.

In order to solve these drawbacks, UCAs have been developed which consist of shell encapsulated droplets. Hall C. S. et al. (2000, J. Acoust. Soc. Am. 108 (6):3049-3057) have studied the efficiency of lipid-encapsulated (non-gaseous) perfluorocarbon emulsion droplets of 250 nm in ultrasound imaging. These particles can be targeted to tissue via antibodies thus providing additional molecular information.

Ideally an ultrasound contrast agent should have as many as possible of the following features:
Stable and sufficient lifetime in blood, e.g. allowing a detection in the targeted organ during 30 minutes or more
A particle size of less than 8 micron, so as to enable them to pass through blood capillaries
Non toxic, or acceptable toxicity
Sufficient reflection enhancement
Ease of production and clinical use
Allowing highly specific targeting

Moreover, the ultrasound contrast agent should preferably be applicable with the existing ultrasound imaging systems, such as the Philips Ultrasound Imaging System.

Different particles comprising metals or metal oxides with magnetic properties have been developed for use as contrast agents for magnetic resonance imaging (MRI). U.S. Pat. No. 5,310,539 describes an image-enhancing agent comprising melanin combined with a non-dissociable signal-inducing metal, wherein the signal inducing metal is preferably a magnetic or paramagnetic metal. US 2002/0136693 describes agents for diagnostic purposes which contain magnetic particles comprising a magnetic double metal oxide/hydroxide or a magnetic metal and optionally a complexing agent. US 2003/0082237 describes nano-particles which are structured into spheres having an inner and outer layer of vesicles by block copolypeptides or homoplymer polyelectrolytes. Either the outer or inner layer of nano-particles can comprise metals or metal oxides, which are optionally functionalized for site-selective medical imaging.

WO 02/11771 describes metal nano-particles clustered on proteins as an ultrasound contrast agent. As several particles are clustered onto one protein, the cluster itself is detected by ultrasound and provides a high background and a low signal to noise ratio. Bekeredjian et al. describe the potential use of gold-bound microtubules as an ultrasound contrast agent (2002, Ultrasound in Med. & Biol. 28(5):691-695). Such gold-bound microtubules displayed longer persistence of contrast activity than conventional contrast agents (microbubbles). However, absolute intensities were generally lower.

The present invention provides a new type of contrast agent which comprises metal nano-particles. These particles when aggregated are acoustic reflectors due to their strong acoustic impedance difference with body tissue and have the advantage over current commercial UCAs (microbubbles) of being stable and that they can be modified in the same way as current targeted contrast agents. It is shown that upon obtaining a film with these nano-particles, a reflection enhancement is obtained. The contrast agent can be used with all forms of sonography, e.g. B-mode, Doppler shift sonography, etc.

Thus, the present invention relates to a contrast agent comprising metal nano-particles having an acoustic impedance above 35.10⁵ g/cm²s, particularly above 50.10⁵ g/cm²s.

More particularly, according to the present invention, the contrast agent comprises metal nano-particles are non-toxic and chemically stable.

More particularly, according to the present invention, the metal nano-particles preferably have a diameter of between 1 nm and 100 nm, particularly between 1 nm and 50 nm.

The contrast agent of the present invention comprises metal nano-particles, which are coated, e.g. by a polymer coating.

The metal nano-particles for use as contrast agents according to the present invention are furthermore targeted, i.e. they comprise a bio-target agent such as cell, tissue, microorganism, e.g. parasite, or biomolecule, e.g. protein, DNA or RNA specific target agents of which antibodies or fragments thereof are only one example.

The metal nano-particles of the present invention can furthermore be used for drug delivery by coating the particles with a therapeutic agent or by including a drug in a coating.

Particular embodiments of the present invention relate to contrast agent comprising metal nano-particles wherein the metal is a non-magnetic metal. According to a further embodiment the metal particles comprise a metal which is a noble metal or a mixture of one or more noble metals, e.g. gold, silver, platinum, palladium, tungsten or tantalum, rhenium. According to a more particular embodiment of the invention, the metal nano-particles are made of gold. Optionally, the metal particles comprise a metal oxide or have a stable thin oxide layer or may have a bio-neutral/biocompatible coating.

Another aspect of the invention relates to the use of the metal nano-particles of the invention as a diagnostic agent, more particularly as an ultrasound contrast agent in ultrasound, e.g. targeted ultrasound contrast imaging. Thus, the invention relates to the use of metal nano-particles having one or more of the above-described characteristics in the production of a contrast agent, for use in ultrasound contrast imaging. This includes the use of the metal nano-particles for the visualization of tissue or parts thereof, as well as their use in the detection of specific targets such as, but not limited to, cellular markers, pathogens, etc.

Moreover, according to a particular aspect of the present invention, the metal nano-particles can also be detected using other imaging means allowing the use of the particles of the invention for combined imaging techniques.

Another aspect of the present invention is a method of diagnosis comprising administration of a contrast agent according to the present invention to an animal or human patient, and performing an ultrasound imaging examination of the animal or human. Alternatively, according to another aspect of the invention the contrast agent is administered to an animal or human tissue for diagnosis ex vivo.

The present invention relates to the use of metal nano-particles in ultrasound contrast agents as well as to the preparation and design of ultrasound contrast agents.

The metal nano-particles according to the present invention have a diameter of between 1-100 nm, preferably less than 50 nm, more particularly 30 nm or less. The shape of the particles is not considered critical or a limitation on the present invention. Any regular (e.g., spherical, polygonal, etc.) or irregular shapes are employable. Similarly, the particle size distribution is not considered critical or a limitation on the present invention although in some applications a certain size range may be of advantage. Different methods have been described for producing nano-particles, including nucleation in solution (i.e. chemical synthesis) and vapor condensation or flame or spray techniques (Gutsch et al. 2002, KONA 20:24-34; Axelbaum, 2001, Powder Metall. 43(3):323-325), but also more recently described techniques of laser ablation, vacuum evaporation on running liquids (VERL), and chemical vapor deposition (CVD) are suitable. Additionally or alternatively, an appropriate-sized nano-particle distribution can be obtained by filtration. Any conventional method for grinding solids to the particle sizes useful in this invention can be employed. According to one embodiment, the production method results in non-aggregating or non-clustering metal nano-particles.

An important characteristic of the metal nano-particles of the present invention is their acoustical impedance, which renders them suitable for use as an ultrasound agent. Acoustic impedance (Z) is defined as the product of density (p) and speed of sound (c) in a medium (Kinsler et al., 1982, Fundamentals of acoustics. 3rd edition, John Wiley and sons, New York). The acoustical impedance of the metal nano-particles of the present invention should be significantly higher than that of body tissues, the acoustical impedance of most body tissues being within the range of 1.3-1.7×10⁵ g/cm²s (with an average of 1.58×10⁵ g/cm²s). The present invention provides that the metal nano-particles of the present invention have an acoustical impedance of at least 35×10⁵ g/cm²s, more particularly at least 50×10⁵ g/cm²s. The maximal acoustic impedance is not a limiting factor of the invention but is envisaged to be around 120×10⁵ g/cm²s.

Examples of metals with an acoustical impedance which is appropriate in the context of the present invention are gold, silver, platinum, palladium, tungsten or tantalum, rhenium, or a mixture thereof, or alloys of metals, such as platinum and iridium. The metals for use in the metal nano-particles are preferably metals which are chemically stable and non toxic or have been rendered chemically stable by an appropriate coating. Of particular interest in this regard are metals that combine the features of appropriate acoustical impedance with stability and limited toxicity. According to one embodiment the metal is a noble metal. According to a particular aspect of the invention, the metal is non-magnetic.

According to a particular embodiment, the metal particles of the invention are essentially solid metal particles, meaning a) that their centre is not hollow and b) that, with the exception of a coating layer described below, they are made up essentially of one or more metals, i.e. formed from a solid metallic core and not associated (with the exception of the outer layer) with non-metal compounds such as proteins, polysaccharides or other structuring compounds, i.e. they are solid metal particles.

According to a particular embodiment of the invention, the metal particles comprise a stable, non-toxic coating, in order to reduce particle aggregation. The coating is preferably bio-neutral and/or biocompatible. Coatings suitable for this end have been described in the art and include natural and synthetic carbohydrates, synthetic polyaminoacids, or physiologically tolerable synthetic polymers (including aptamers) and derivatives thereof.

Carbohydrates include natural and synthetic structural polysaccharides such as pectins and pectin fragments such as polygalacturonic acid, the glycosaminoglycans and heparinoids (e.g. heparin, heparan, keratan, dermatan, chondroitin and hyaluronic acid), dextrans, celluloses and the marine polysaccharides such as alginates, carrageenans and chitosans, and their derivatives.

Synthetic polymers that can be used as coatings include but are not limited to polyaminoacids, polyacrylates, and polystyrenes. Among the polyaminoacids homo- and copolymers of lysine, glutamic acid and aspartic acid and their esters (eg methyl and ethyl esters) are non-limiting examples of the envisaged coating. Moreover, coating with multiblock copolymers is also envisaged, such as multiblocks of polylactic acid (PLA), polyglycolic acid (PGA), polyanhydrides, polyphosphazenes or polycaprolactone (PCL). According to a particular embodiment, the metal nano-particles are provided with a combination of different coatings.

Alternatively, the metal particles can be coated with a stable thin oxide layer, provided that this layer is non-toxic. Such a coating impart a charge to the particles resulting in an electrical repulsion between the particles However, such a coating is particularly suited for use of the metal nano-particles in tissues with low ion concentrations, as ions will reduce the electrical repulsion causing agglomeration of the particles (e.g. in blood).

Coating agents according to the present invention may contain reactive functional groups such as amine, active ester, alcohol, and carboxylate. Such functional groups may be used to attach onto the surface of the particles biologically active molecules, especially bio-target specific agents. Suitable bio-target specific agents may be cell-, microorganism-, e.g. parasites such as nematodes or bacteria-, organ- or tissue specific molecules such as peptides or proteins, or antibodies or fragments thereof. Included within the term bio-target specific agents are molecules or functional groups directed at a specific foreign and/or toxic agent. The coating may also comprise molecules affecting the charge, lipophilicity or hydrophilicity of the particle or its ability to enter through a cell membrane.

A particular embodiment of the present invention relates to metal nano-particles which are targeted to a particular organ or tissue. This can be achieved by attaching to the surface of the nano-particle a tissue or organ-specific molecule. One such molecule is an antibody, directed against an organ or tissue-specific antigen. For instance, such antibody can be a polyclonal or monoclonal antibody specific for a tumor-associated antigen or antimyosin. Non-limiting examples of polyclonal or monoclonal antibodies which can be used for conjugation include, especially, those that are principally directed at antigens found in the cell membrane. For example, suitable for the visualization of tumors are polyclonal or monoclonal antibodies per se, and/or their fragments (Fab, F(ab)₂), which are directed, for example, at the carcinoembryonal antigen (CEA), human choriogonadotrophin (.beta.-hCG) or other antigens found in tumors such as glycoproteins. Antimyosin, anti-insulin and antifibrin antibodies and/or fragments, inter alia, are also suitable. Alternatively, the molecule is a ligand for a receptor with a tissue-specific expression pattern. In the context of the present invention the term ‘cellular marker’ is used to refer to any molecule which allows the identification of a specific cell, celly-type, tissue, type of tissue, organ or type of organ.

A further particular embodiment of the present invention relates to particles which are coated with a drug, or which have a drug incorporated in a coating, for use as drug-delivery agents or for combined diagnostic and therapeutic use. Therapeutic agents can be selected over a wide range of drugs and are determined by the therapeutic target.

Optionally the particles are further coated with a material that provides them with a hydrophilic coating to minimize the uptake of blood components and/or a steric barrier to particle-cell interaction, in order to minimize uptake by the liver. An example of such a material is the block copolymer known as tetronic 908 (U.S. Pat. No. 4,904,497).

The contrast agents can be used for all applications of sound waves in therapy and diagnosis, e.g. Doppler shift, or B-mode sonography. As described in U.S. Pat. No. 6,165,440, ultrasonic waves can be used to obtain perforation of tumor blood vessels, microconvection in the interstitium, and/or perforation of cancer cell membrane. Following this principle, the coated metal nano-particles of the present invention can be used to obtain enhanced delivery of macromolecular therapeutic agents into cancer cells with minimal thermal and mechanical damage to normal tissues.

Particles of this invention are optionally formulated into diagnostic compositions for enteral or parenteral administration. For example, parenteral formulations advantageously contain a sterile aqueous solution or suspension of coated metal particles according to this invention. Various techniques for preparing suitable pharmaceutical solutions and suspensions are known in the art. Such solutions also may contain pharmaceutically acceptable buffers and, optionally, additives such as, but not limited to electrolytes (such as sodium chloride) or antioxidants. Parenteral compositions may be injected directly or mixed with one or more adjuvants customary in galenicals (e.g., methyl cellulose, lactose, mannite) and/or surfactants (e.g., lecithins, Tweens.RTM., Myrj.RTM.).

Conventional excipients are pharmaceutically acceptable organic or inorganic carrier substances suitable for parenteral, enteral or topical application which do not deleteriously react with the agents. Suitable pharmaceutically acceptable adjuvants include but are not limited to water, salt solutions, alcohols, gum arabic, vegetable oils, polyethylene glycols, gelatine, lactose, amylose, magnesium stearate, talc, silicic acid, viscous paraffin, perfume oil, fatty acid monoglycerides and diglycerides, pentaerythritol fatty acid esters, hydroxy-methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colouring, flavouring and/or aromatic substances and the like which do not deleteriously react with the active compounds.

Formulations for enteral administration may vary widely, as is well-known in the art. In general, such formulations include a diagnostically effective amount of the metal particles in aqueous solution or suspension. A syrup, elixir or the like can be used wherein a sweetened vehicle is employed. Alternatively, the formulation can be in tablets, dragees, suppositories or capsules having talc and/or a carbohydrate carrier or binder or the like, the carrier preferably being lactose and/or corn starch and/or potato starch.

For parenteral application, particularly suitable are injectable sterile solutions, preferably oil or aqueous solutions, as well as suspensions, emulsions, or implants, including suppositories. Ampoules are convenient unit dosages. The contrast agents containing metal nano-particles are preferably used in parenteral application, e.g., as injectable solutions.

The diagnostic compositions of this invention are used in a conventional manner in ultrasound procedures. The diagnostic compositions are administered in a sufficient amount to provide adequate visualization, to a warm-blooded animal either systemically or locally to an organ or tissues to be imaged, then the animal is subjected to the medical diagnostic procedure. Such doses may vary widely, depending upon the diagnostic technique employed as well as the organ to be imaged.

The contrast agents of this invention generally contain from 1 micromole to 1 mole, preferably 0.1 to 100 millimoles of metal per liter and are usually dosed in amounts of 0.001 to 100 micromoles, preferably 0.1 to 10 micromoles of metal per kilogram of body weight. They are administrable enterally and parenterally to mammals, including humans. Typically, diagnostic measurement is begun about 5-30 minutes after administration.

According to a specific embodiment of the present invention, the diagnostic composition of the invention are used for the imaging, i.e. the visualization of a tissue structure or target molecule in a tissue sample or organ ex vivo, i.e. on a tissue sample or organ that has been completely or partially isolated from the animal or human body.

The use of the contrast agents of the present invention are envisaged in a wide range of applications, including all applications which have been described for contrast imaging in the art, such as, but not limited to visualization and diagnosis of tissues, parts thereof or structures therein (e.g. as tracers). For instance, contrast imaging is used in the visualization of the cardiovascular system (e.g. wall motion analysis, myocardial perfusion, identifying areas of infarction or ischemia in the myocardium, identifying blood clots) or the liver (liver function, detection of liver tumors). Other applications of contrast imaging envisaged include, but are not limited to visualization of the gastro-intestinal tract, visualization of tumors, identifying testicular and ovarian torsion, evaluation of renal and other transplanted organs, remote measurement of temperature, physiological pressure, and contrast agent-guided and controlled local drug delivery.

According to one aspect, the diagnostic compositions of the invention are used for combined use in different imaging methods. Thus, depending on their characteristics, the metal nano-particles of the invention may be appropriate for use in X-ray analysis. Thus, a particular embodiment of the invention relates to a diagnostic composition for use in combined imaging methods.

As used herein “comprising” is to be interpreted as specifying the presence of the stated features, integers, steps or components as referred to, but does not preclude the presence or addition of one or more features, integers, steps or components, or groups thereof. Reference herein to ‘a’ or ‘an’ does not exclude a plurality.

The following Examples, not intended to limit the invention to specific embodiments described, may be understood in conjunction with the accompanying Figure, incorporated herein by reference, in which:

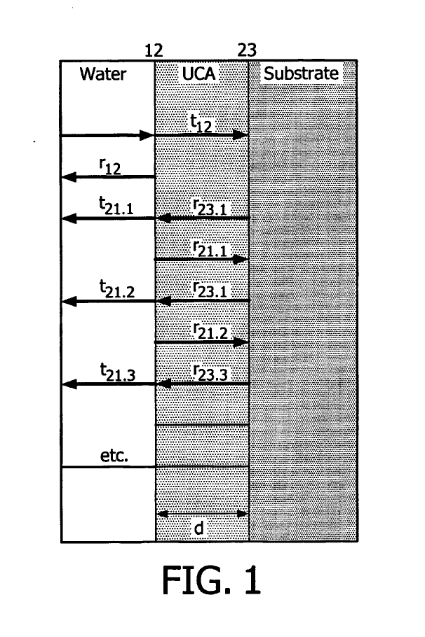

FIG. 1: Illustration of the parameters used in the theoretical model for reflection enhancement (of an incompressible layer).

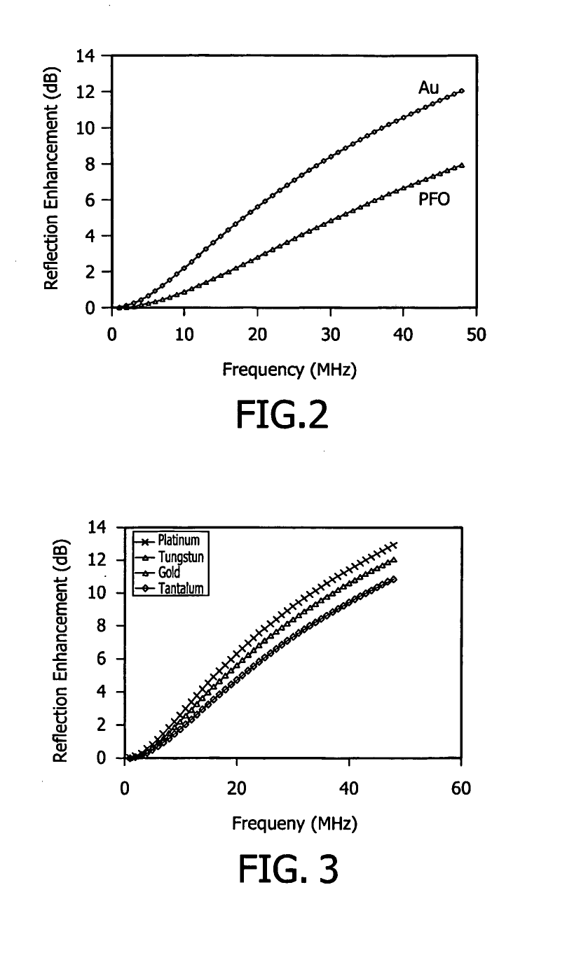

FIG. 2: Theoretical calculated reflection enhancement of a 50 nm Au layer versus a 250 nm liquid-perfluorocarbon layer (PFO) on top of material with the same acoustic properties as average human tissue as a function of the frequency.

FIG. 3: Theoretical predicted reflection enhancement of a 50 nm platinum layer (×), a 50 nm tungsten layer (Δ), a 50 nm gold layer (-Δ-) and a 50 nm tantalum layer (⋄) as a function of the frequency.

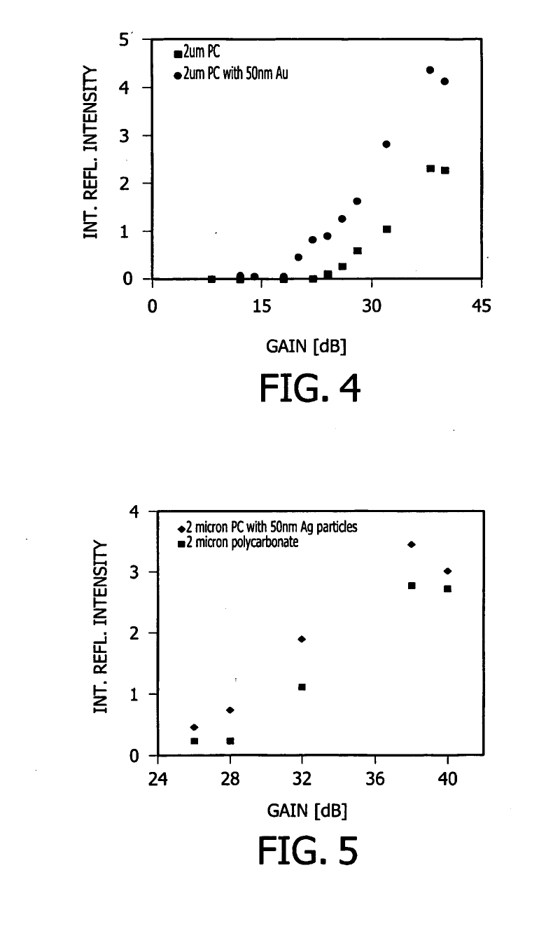

FIG. 4: The integrated reflected intensity (peak area) of a 2 um PC foil and a 2 um PC foil with 50 nm evaporated Au as a function of the gain.

FIG. 5: Integrated reflected intensity (area of the peak) as a function of the gain (the intensity in dB which is generated by the transducer).

### EXAMPLES

### Example 1

### Theoretical Calculation of the Reflection Enhancement of a Gold Film vs Perfluorocarbon Emulsion Droplets

The reflection enhancement of a layer can be calculated using a mathematical model:

Wherein:

‘r(k)’ is the amplitude reflection coefficient of incompressible materials,

‘t’ is the complex transmission coefficients between medium 1 e.g. water, medium 2 the ultrasound contrast layer/agent and medium 3 e.g. the substrate,

‘r’ is the complex reflection coefficients between medium 1 e.g. water, medium 2 the ultrasound contrast layer/agent and medium 3 e.g. the substrate (see FIG. 1),

‘k’ is the wave number of the ultrasonic wave in the contrast layer,

‘d’ is the thickness of the contrast layer.

And the enhancement is 20.log.(|r(k)|/r₀|)

‘r(k)’ is the amplitude reflection coefficient of incompressible materials, r₀ is the amplitude reflection coefficient of the substrate surface without the contrast agent.

The enhancement as calculated for a layer of perfluorocarbon emulsion droplets of 250 nm was in reasonable agreement with an ultrasound reflection enhancement observed for a layer of such particles on material with the acoustic properties of spleen tissue (1.6×10⁵ g/cms), of which the acoustical impedance is very close to the average acoustical impedance of human tissue (1.58×10⁵ g/cms).

FIG. 2 shows the theoretical calculated reflection enhancement of a 50 nm Au layer versus a 250 nm liquid-perfluorocarbon, lipid-encapsulated nanoparticulate emulsion layer (PFO) on top of human cloth, as a function of the frequency.

It can be concluded that the reflection enhancement of a 50 nm Au layer is higher than that obtained by a 250 nm liquid-perfluorocarbon, lipid-encapsulated nanoparticulate emulsion layer.

### Example 2

### Theoretical Predicted Reflection Enhancement of a 50 nm Platinum Layer, a 50 nm Tungsten Layer, a 50 nm Gold Layer and a 50 nm Tantalum Layer as a Function of the Frequency

FIG. 3 shows the theoretical predicted reflection enhancement of a 50 nm platinum layer, a 50 nm tungsten layer, a 50 nm gold layer and a 50 nm tantalum layer as a function of the frequency.

### Example 3

### Measurement of the Reflection Enhancement of a 50 Nanometer Gold Layer

50 nanometer of gold was evaporated on a polycarbonate (PC) foil of 2 micrometer. A Digital Ultrasound Imaging System of Taberna Pro Medicum equipped with a 22 MHz transducer was used to measure the reflection of the PC foil with and without the evaporated gold layer. The integrated reflected intensity (peak area) of a 2 um PC foil and a 2 um PC foil with 50 nm evaporated Au as a function of the gain is shown in FIG. 4.

50 nm evaporated gold on top of a 2 micrometer polycarbonate foil gives a reflection enhancement of 4 dB.

### Example 4

### Measurement of the Reflection Enhancement of a Silver Nano-Particle Layer

A polycarbonate (PC) foil of 2 micrometer was coated with a 50 nm layer of silver particles, the particles having a size of 30 nm. A Digital Ultrasound Imaging System of Taberna Pro Medicum equipped with a 22 MHz transducer was used to measure the reflection of the PC foil with and without the Ag layer. The result is shown in FIG. 5.

It can be concluded that the 50 nm layer of silver nano-particles increases the reflectivity of a PC foil 2.5 times. Thus, the film of metal nano-particles provides a significant enhancement demonstrating the utility of these particles as ultrasound reflectors.

1. A contrast agent comprising solid metal nano-particles having an acoustic impedance above 35.10⁵ g/cm²s.

2. The contrast agent of claim 1, having an acoustic impedance of above 50.10⁵ g/cm²s.

3. The contrast agent of claim 1, wherein said metal nano-particles have a diameter of between 1 nm and 100 nm.

4. The contrast agent of claim 1, wherein said metal nano-particles have a diameter of between 1 nm and 50 nm.

5. The contrast agent of claim 1, wherein said metal is non-magnetic.

6. The contrast agent of claim 1, wherein said metal is selected from the group consisting of gold, silver, platinum, palladium, tungsten or tantalum, rhenium, or a mixture thereof.

7. The contrast agent of claim 1, wherein said metal is a noble metal.

8. The contrast agent of claim 1, which further comprises one or more coatings.

9. The contrast agent of claim 8, wherein said coating comprises natural or synthetic carbohydrates, synthetic polyaminoacids, or physiologically tolerable synthetic polymers or derivatives thereof.

10. The contrast agent of claim 8, wherein said one or more coating comprises a therapeutic agent.

11. The contrast agent of claim 1, characterized in that one or more bio-target-specific molecules are attached to the surface of said metal particle.

12. The contrast agent of claim 11, wherein said bio-target-specific molecule recognizes a target which is selected from the group consisting of a cellular marker, a pathogen and a foreign and/or toxic agent.

13. The contrast agent of claim 11, wherein said bio-target-specific molecule is an antibody or a fragment thereof.

14. Use of a metal nano-particle according to claim 1 in the manufacture of an ultrasound contrast agent.

15. A method of diagnosis comprising administration of a contrast agent according to claim 1 to an animal or human patient, and performing an ultrasound imaging examination of the animal or human.

16. A method of imaging an isolated tissue sample or organ, which method comprises administrating the contrast agent according to claim 1 to said tissue sample or organ and performing an ultrasound imaging examination thereof.
